(19) United States
(12) Patent Application Publication
(10) Pub. No.: US 2016/0058888 A1
(43) Pub. Date: Mar. 3, 2016

(54) Treatment of Operable High-Grade Glioma With Sitimagene Ceradenovec Gene Therapy and Ganciclovir

(71) Applicants: FINVECTOR VISION THERAPIES LIMITED, Chinnor, Oxfordshire (GB); PHARMACEUTICAL PATENT ATTOENEYS, LLC., Morristown, NJ (US)

(72) Inventor: Seppo Yla-Herttuala, Kuopio (FI)

(73) Assignee: Gliotherapy Limited, Chinnor (OX)

(21) Appl. No.: 14/787,887

(22) PCT Filed: May 8, 2014
(86) PCT No.: PCT/US14/37238

§ 371 Date: Oct. 29, 2015

Related U.S. Application Data
(60) Provisional application No. 61/820,869, filed on May 8, 2013.

(51) Int. Cl. A61K 48/00; A61K 31/522; A61K 38/45; A61K 31/495

(52) U.S. Cl. CPC A61K 48/00; A61K 31/495; A61K 31/522; A61K 38/45

(57) ABSTRACT

Gene therapy with genes for prodrug converting enzymes adds to local control of glioblastoma achieved by surgery. There appears to be a pronounced local reaction which is in part inflammatory and has a measurable immunological component. Considering the widely proven fact that during the weekslong gap-phase between surgery and completion of radiation or chemoradiation tumour growth from the infiltrative zone may continue unhampered, a treatment such as SIT will cover that time period.

### RELATED APPLICATIONS

This application is the PCT national stage entry of PCT Application Serial No. PCT/US2014/037238, filed 8 May 2014, which in turn asserts priority from United States Provisional patent filing Ser. No. 61/820,869, filed 8 May 2014, each incorporated here by reference.

### BACKGROUND

Apart from progress in the treatment of adult glioblastoma with temozolamide and radiation in patients with favourable methylation status, local or less frequently distal recurrence and progression is the natural course of the disease. Local control of the disease by complete removal adds to time to progression and survival. Adenovirus mediated gene therapy with a prodrug converting enzyme (AdvHSV-TK, sitimagene ceradenovec (SIT) Cerepro) which is locally applied in patients with newly diagnosed resectable glioblastoma followed by intravenous ganciclovir (GCV) has shown efficacy in phase II studies and therefore further clinical evaluation was warranted.

Malignant gliomas comprise more than half of all gliomas and are the most common primary brain tumours. Despite multi-modal therapy involving surgery, radiotherapy and chemotherapy, the prognosis for malignant gliomas is poor with the average survival of unselected operated patients with glioblastoma around one year¹. As there is a positive correlation between local control achieved by radiologically complete resection and survival^{2 3} improvement of local control beyond the margin of resection should be a beneficial element of the multimodal treatment of glioblastoma which includes resection, radiation and chemotherapy as current standard⁴.

Treatment of the peritumoural region is directed at tumour cells which have moved away from the bulk and are invariably present even after gross total resection in the so-called invasive zone. While carmustine wafers are the only approved local intracavitary therapy⁵, its efficacy is limited and diffusion of the active compound is variable and depends on the extent of oedema. Other approaches have included in the past convection enhanced delivery with toxin conjugates⁶ and also an early attempt of gene therapy with retrovirus packaging cells transducing the gene for a prodrug converting enzyme, herpes simplex virus thymidine kinase (HSV-Tk)⁷. This approach was limited by its very low rate of transduction due to the application method which was by vector producing cells with limited viability and low virus production.

Local therapies which can be applied during surgery have the potential to begin to work on residual tumour cells immediately or early after resection without the necessity to wait for wound healing and the scheduling for radiation which can take up to four weeks depending on internationally highly variable capacities. With little progress in this important field, however, local gene therapy was re-evaluated with an improved agent. Using an adenoviral vector with high titre, adenovirus-mediated Herpes-Simplex virus thymidine kinase (HSV-tk) gene therapy administered locally intraoperatively in conjunction with subsequent ganciclovir (GCV), has been developed for the treatment of operable high-grade glioma. The active agent, sitimagene ceradenovec (SIT) (Cerepro®, Ark Therapeutics Ltd, London, UK) is a first generation replication-deficient adenovirus (serotype 5 with E1 with partial E3 deletions), and containing the cDNA for HSV-tk. Expression of the transgene results in the production of the enzyme thymidine kinase (tk), which is able to phosphorylate ganciclovir (GCV), leading to the production of ganciclovir triphosphate, a cytotoxic nucleotide analogue that “selectively” kills dividing cells by being incorporated into DNA leading to apoptosis, both in transduced cells and adjacent dividing cells through a “bystander” effect^{8,9,10}. From preclinical studies it is known to transfect glioma and non malignant glial cells^{11,12,}, but normal neurons surrounding the tumour do not usually proliferate and therefore are not susceptible to the toxic effects of GCV metabolites.

Clinical experience with SIT has shown very promising efficacy in three phase I/II studies using AdvHSV-TK. In the first two phase I studies optimal dose (1×10¹² vp) and transduction efficiency were determined^{13,14}. Also, the treatment was compared to HSV-TK retroviral packaging cells¹³. It was concluded that AdvHSV-TK showed promising results and that several small injections covering as much as possible of the surface area of the tumour cavity are needed to maximize the treatment effect. In a following randomized, controlled phase II trial, safety and efficacy of the treatment were demonstrated by showing a significant prolongation of survival as defined by time to death or surgery for recurrence¹⁵.

Based on these data and significant functional improvements on transgene delivery, a randomised, open label, parallel group multicenter phase III trial was designed to investigate the efficacy and safety of SIT with subsequent GCV for the treatment of patients with operable newly diagnosed glioblastoma compared to standard treatment. We here provide the full report of the study.

### BRIEF DESCRIPTION OF THE FIGURES

FIG. 1 MR Images showing the immediate postoperative image on the left and the same patient 19 days post OP after receiving intravenous ganciclovir for 14 days on the right. The increase in perilesional contrast enhancement in T1 subsequently resolved.

FIG. 2 Study design flow chart illustrating the flow from 256 screened patients to the 236 patients which were finally evaluated. The “loss” of 20 patients is due to the fact that patients are entered into the study before definitive histological diagnosis.

FIG. 3 Kaplan-Meyer Plot for the whole study population in relation to the primary endpoint. (o=censored observation)

FIG. 4A and 4B Kaplan-Meyer Plots for the available non-methylated subgroups of patients showing the result for the primary endpoint which was time to death or re-intervention reaching a HR of 1·72, (95% Cl 1·15-2·56) p=0·0007, (upper panel) and the secondary endpoint all cause mortality with a HR=1·42, (95% Cl 0·92-2·12) and p=0·11 (lower panel).

FIG. 5 As described in the methods section anti-adenoviral antibodies were measured over time and coinciding at the day 19 time point when also an MRI was obtained, the measurable reaction in the serum coincided with contrast enhancement as seen on MRI.

FIG. 6. Tumour sizes of all patients were captured at the time of re-intervention showing that the tumour sizes of the SIT group were similar as in the control group.

### DETAILED DESCRIPTION

We performed an international, open label, randomised, parallel group multicenter phase III clinical trial investigating efficacy and safety of locally derived adenovirus mediated gene therapy with a pro-drug converting enzyme (ASPECT). Patients were randomised before surgery to receive surgical resection of the tumour and intraoperative perilesional injection of SIT (1×10¹² viral particles) followed by GCV (postoperatively, 5 mg/kg IV twice daily) in addition to standard care or resection and standard care alone. Temozolomide not being standard in all participating countries at the time of the study was allowed at the discretion of the treating physician. The primary objective determined whether SIT/GCV was superior to standard care, with time to death or re-intervention as the primary endpoint. Other assessments included time to all cause mortality, safety and tolerability.

Overall, 251 patients were randomised and 236 patients met the ITT criteria for the study, (119/124 SIT, 117/126 Standard Care). Baseline characteristics were comparable between groups. Accounting for actual temozolamide use in a statistical analysis which included temozolamide as a time dependent covariate, we demonstrated a significant benefit for the primary endpoint (HR=1·53, 95% Cl 1·13-2·07, p=0·006) which even became more apparent in the subgroup with MGMT non-methylated status (HR=1.72, 95% Cl 1.15-2.56, p=0.008).

The safety profile of the whole study is influenced by the fact that including surgery as part of the study, any surgical morbidity is captured on top of agent related events. It was found that safety was mainly related to events of the local injections of vector, and included hemiparesis (which was mostly transient) but also overall survival (HR=1.18 (0.86-1.61, p=0.31), but treatment had a significant effect on time to survival.

SIT appears to be beneficial as a supplementary treatment of resectable glioblastoma and delays time to pharmacological or surgical reintervention. The adverse event profile appeared manageable and acceptable and as expected for local therapy for patients with glioblastoma.

### Methods

### Adenovirus HSV-TK

AdvHSV-TK (Cerepro®, sitimagene ceradenovec) is based on a first generation E1, partial E3 deleted serotype 5 adenoviral vector¹⁵. The HSV-TK cDNA was cloned under a CMV promoter and the structure was verified by sequencing. Clinical lots of the virus were produced by amplification of a Master Viral Seed Stock in adherent HEK293 cells by Ark Therapeutics Oy (Kuopio, Finland). The required toxicology studies were conducted¹⁶ on material from this process prior to obtaining regulatory approval for the study Drug product was formulated at a concentration of 1×10¹² viral particles (vp/ml) in 5 mM Hepes, pH 7.8, containing 20% (v/v) glycerol. Prior to administration, the virus (total dose 1×10¹² vp) was diluted in physiological saline to a volume of 10 ml.

### Patients

Adult patients, aged 18-70 years (Karnofsky score ≧70 at screening), with newly diagnosed supratentorial glioblastoma multiforme (GBM) which appeared to be amenable to complete resection were eligible for the trial thus excluding bihemispheric or multifocal tumours. The usual exclusion criteria were applying specifically allergy to gancyclovir.

The study was performed at 38 sites: 33 in Europe and five in Israel. The protocol was approved by local independent ethics committees and all subjects gave their written informed consent prior to any trial-related procedure. The trial (EudraCT Number: 2004-000464-28) was performed in accordance with the principles of Good Clinical Practice and the declaration of Helsinki. Neither the patient nor the investigator was blinded to the treatment during the course of the study.

### Procedures

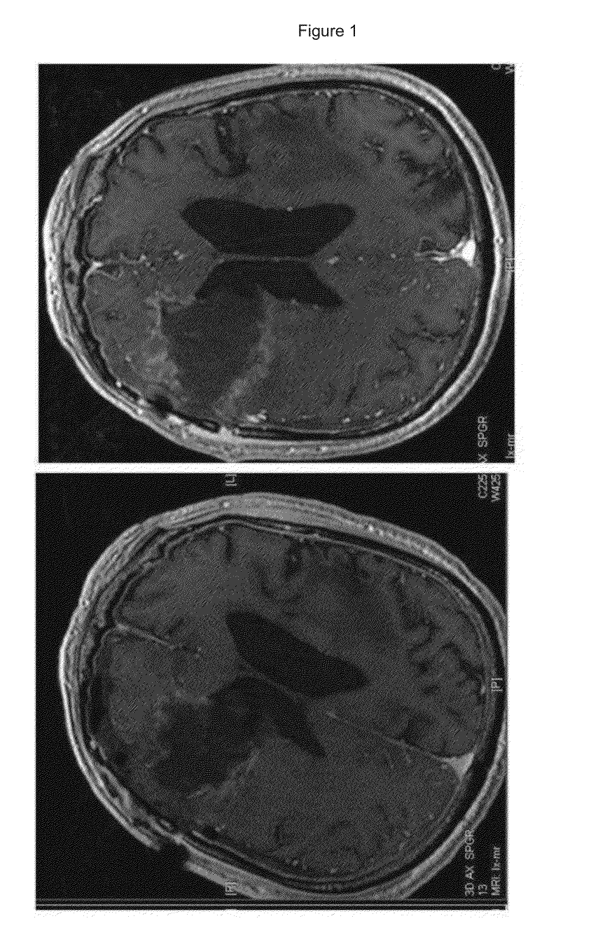

Patients were randomised (1:1) the day before surgery to either SIT or Standard Care. The SIT group received a one-time treatment of SIT administered as a series of injections (between 30 and 70) introducing 1×10¹² vp into the wall of the resection cavity at the end of the completed resection, using a blunt needle which was advanced up to 2 cm (tissue depth permitting) slowly administering 100 ul per injection site which could later be seen on MRI as little cavitations (FIG. 1). After allowing for transduction for five days, GCV (from day 5 to 19 postoperatively) was given 5 mg/kg IV twice daily (Roche Pharmaceuticals, Welwyn Garden City, UK).

Standard care represented during the time of the study was still heterogeneous as for the general acceptance, use and reimbursement for temozolamide in an international setting. Surgery and radiotherapy (60 Gy in 30 fractions) was seen as the standard and radiochemotherapy according to the Stupp protocol⁴ as an option. The first patient was randomised in November 2005. As the study progressed, temozolomide was becoming more frequently, though not universally, used. A protocol amendment (December 2005) allowed the use of temozolomide, following surgery, at the discretion of the investigator. Use of temozolamide was captured in the case report forms of the patients. The last patient was randomised in April 2007. After the end of the formal study, all surviving patients were to be followed up at least annually for their outcome.

### Laboratory Analyses

Methylquanine-methyltransferase (MGMT) promoter methylation analysis: Genomic DNA was isolated from glioblastoma tissue (formalin fixed paraffin embedded tissue or fresh frozen tissue) and used for methylation-specific PCR of MGMT and β-Actin (reference gene) promoter regions. Both promoter regions were assessed and quantified in parallel and the ratio of the two calculated (performed by Oncomethylome Sciences B.V., Leiden, Netherlands)¹⁷.

AdV Antibody analysis: Neutralising antibodies to serotype 5 adenovirus were measured in serum prior to surgery and at intervals post surgery (Bioreliance, Glasgow, UK). Serial dilutions of patient serum were incubated with Cerepro vector before exposure to HEK 293 detector cells. The antibody titre is the reciprocal of the dilution of serum that reduces the virus infectivity of a spike of 10 TCID₅₀/well by 10-fold thus resulting in 50% of the assay wells exhibiting CPE in this assay.

Analysis for systemic vector spread: Quantification of vector DNA in blood was performed using QPCR (Covance Laboratories, Harrogate, UK) at intervals after surgery. The sequences of the primers are as follows: forward, GGCTAACTAGAGAACCCACTGCTT; reverse, AAGCCATACGCGCTTCTACAA; probe, TGGCGTGAAACTCCCGCACCTC. 1 μg of extracted DNA (QIAamp® DNA Blood Mini Kit) was used per replicate where possible. The detection system used was the ABI PRISM 7700™ (Applied Biosystems, US). The limit of quantitation was 10 copies per reaction.

Central imaging analysis was performed according to a pre-specified Imaging Evaluation Plan (IEP) by Bio-Image Technologies S.A.S. (Lyon, France office) collecting MRI obtained with a standardized volumetric protocol with and without contrast at diagnosis, early postoperatively (within 48 hrs) and then on days 19, and month 3 and every three months thereafter. Based on a proprietary 3D image registration algorithm (BioImage) enhancing tumour volumes were assed discounting hemorrhage, cysts and necrosis. Because of an unexpected increase of enhancement at day 19 in the experimental arm, further assessment of these scans in a blinded manner by members of the Steering Committee, suggested this observation was probably due to an injection related “pseudo-progression” which has been described in another context and involves an appearance of an increase in the tumour size but this then regresses spontaneously¹⁸. This was taken into account in an adapted, second definition of MRI progression in the IEP by stipulating that an increase in tumour volume must be seen at two consecutive timepoints and the independent assessment must state that tumour progression had occurred at the two consecutive timepoints. However, if a re-intervention occurred in this time then the tumour progression was regarded as ‘true’ progression and the above rule was not applied.

### Statistical Analysis

The pre-specified primary endpoint for this study was time to death or re-intervention, defined as any kind of treatment (including surgery, radiotherapy or chemotherapy) given to prolong survival when a tumour recurred or progressed. This composite endpoint was chosen to take into account that this was an open study and patients in the standard treatment arm were likely to seek experimental treatment at progression and such a composite endpoint reflecting efficacy in a disease stage had been used successfully in other studies such as cardiological intervention trials¹⁹. A secondary outcome measure was time to all cause mortality. Safety and tolerability of SIT was also assessed.

Patients meeting the entry criteria for the study were randomised within 24 hours prior to the planned surgery, in blocks of four. A single randomisation schedule was generated, prior to any patient entering the study.

The intent-to-treat (ITT) population was used in efficacy and all randomised patients for safety analysis. The ITT population was defined as all randomised patients who had a glioma (low or high grade) as confirmed by central histology review (Prof. James W., Ironside, Department of Neuropathology, Western General Hospital, Edinburgh, UK). According to the initial statistical analysis plan for the ITT population, all patients were to be analysed according to the randomised treatment regardless of actual treatment received.

The trial recruited 251 patients and the pre-specified primary analysis was a triangular test²⁰ using the log-rank test adjusted for intent to use temozolomide and based on the intent-to-treat (ITT) population. It comprised a series of interim analyses which were seen only by an independent data safety monitoring board (DSMB, see acknowledgement): the first, 12 months after the first patient randomised, and subsequent interim analyses at 6 monthly intervals. Each interim analysis was based on a log-rank statistic, Z, stratified for intended temozolomide use as specified at the time of patient randomization. In accordance with this prespecified evaluation paradigm which after all did not reflect study reality, it was recommended at the third interim analysis to stop the study by the DSMB due to futility. Final visits were performed for all surviving patients.

Due to changing practice in the use of temozolomide during the trial and the introduction of MGMT promoter methylation analysis which was noted to be a major prognostic factor²¹, a COX's proportional hazards model including temozolomide as a time-dependent covariate and MGMT methylation status as a fixed covariate were used.

Kaplan-Meier survival curves were plotted by treatment group for the primary endpoint and for all-cause mortality. All-cause mortality was also analysed using Cox's proportional hazards model including actual temozolomide use and MGIT status. Additional covariates examined for both the primary endpoint and all-cause mortality were age, Karnofsky score at baseline and extent of surgical resection²².

This study was sponsored by Ark Therapeutics. Ark Therapeutics and the academic investigators were responsible for the design, execution of the study. Data collection, monitoring and initial statistical analysis were by Covance (Maidenhead, UK) and subsequent analysis by Quanticate (Hitchin, UK) and entered into the sponsor's data management systems and were analysed by the sponsor's statistical team. All authors contributed to the interpretation of data and subsequent writing, reviewing, and amending of the report; the first draft of the report was prepared by the first author, in cooperation with the members of the steering committee and the sponsor. All authors vouch for the accuracy and completeness of the reported data and attest that the study conformed to the protocol and statistical analysis plan.

All authors had full access to all of the data. The corresponding author had the final responsibility for submission for publication of this study. The decision to submit this paper for publication was taken by the Steering Committee for the study (MW, PM, PW and ZR).

### Results

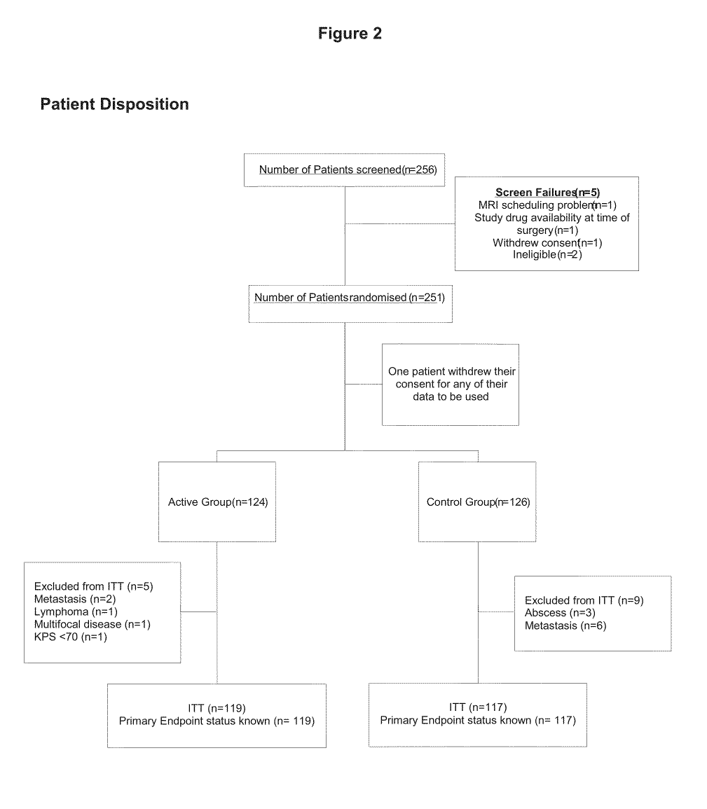

From November 2005 until April 2007, 251 patients were randomised to SIT (n=124) or Standard Care (n=126) in 38 centers in 9 countries Of these, 250 were analysed for safety (one patient withdrew consent for any data to be used), 241 had a known primary endpoint status, and 236 were included in the ITT population (FIG. 2). Patients were excluded from the ITT population due to eight having metastatic tumour, one died before operation, one had bilateral tumour, four patients turned out not to have high grade glioma and one withdrew consent.

Demographic data such as age, gender and the tumour histology were well balanced between the groups (Table 1) although there were some minor differences with respect to Karnofsky score.

TABLE 1
Demographics and Baseline Characteristics*

| | Active Group | Control Group |
|---|---|---|
| Characteristic | (n = 119) | (n = 117) |
| Age, mean (SD) | 55.8 (10.28) | 55.1 (9.90) |
| Male (%) | 60 | 65 |
| Histopathology diagnosis (n %) | | |
| GBM | 112 (94.1) | 111 (94.9) |
| Other high grade glioma | 4 (3.4) | 4 (3.4) |
| Other | 3 (2.5) | 2 (1.7) |
| Karnofsky score (n %) | | |
| 70 | 18 (15.1) | 11 (9.4) |
| 80 | 22 (18.5) | 23 (19.7) |
| 90 | 49 (41.2) | 47 (40.2) |
| 100 | 30 (25.2) | 36 (30.8) |
| Resection during surgery (n %) | | |
| Radical | 99 (83.2) | 95 (81.2) |
| Partial | 20 (16.8) | 22 (18.8) |
| Extent of tumour resected (n %)† | | |
| <50% | 2 (1.7) | 3 (2.6) |
| 50-69% | 5 (4.2) | 8 (6.8) |
| 70-89% | 30 (25.2) | 22 (18.8) |
| ≧90% | 80 (67.2) | 80 (68.4) |
| Not Done | 2 (1.7) | 4 (3.4) |

*ITT population.
†Estimated from postoperative magnetic resonance imaging

No important differences in respect to co-morbidities or past medical histories were present between the patients in the respective treatment arms at baseline.

### Efficacy

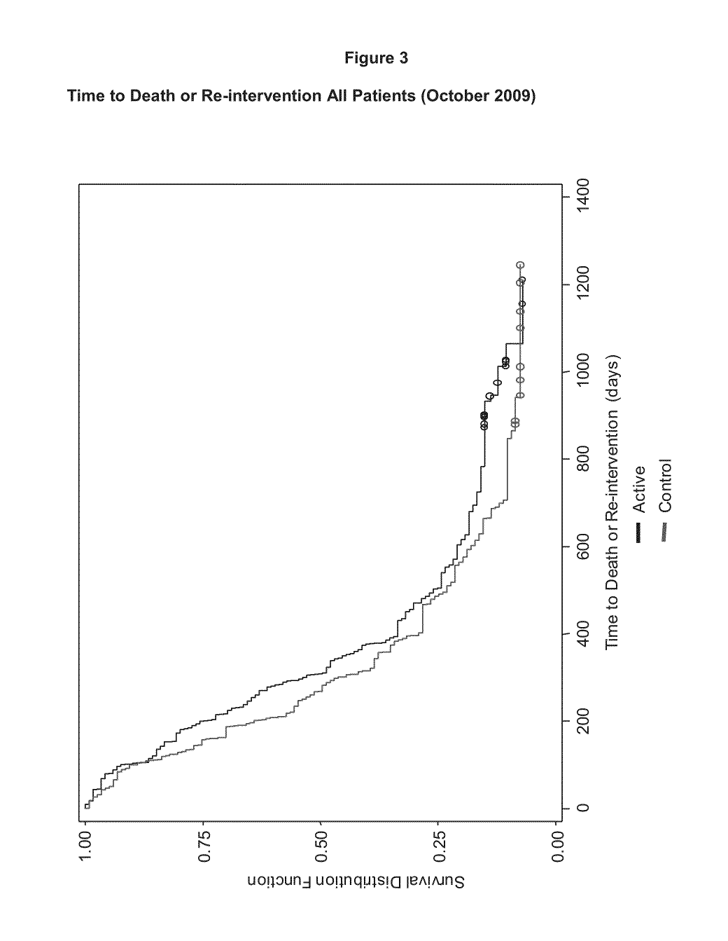

A Cox's proportional hazards model for the primary endpoint including terms for treatment, age, extent of resection, baseline Karnofsky score, MGMT status and temozolomide use as a time dependent covariate was undertaken. Overall, the actual use of temozolomide was not equally distributed between the groups (49% SIT and 65% Standard Care patients). In this analysis, SIT significantly improves the outcome in terms of the primary endpoint (HR 1·44, 95% Cl 1·05-1·99, p=0·024). A further analysis of the outcome on data gathered in October 2009 confirmed this effect (HR 1·53, 95% Cl 1·13-2·0, p=0·0057). The Kaplan Meier survival curves for the primary endpoint are shown in FIG. 3. The effect of SIT on overall survival appeared to be less than on the primary endpoint (HR 1·18, 95% Cl 0·86-1·61, p=0·31). The 2 year survival was 25% in the SIT group and 21% in Standard Care.

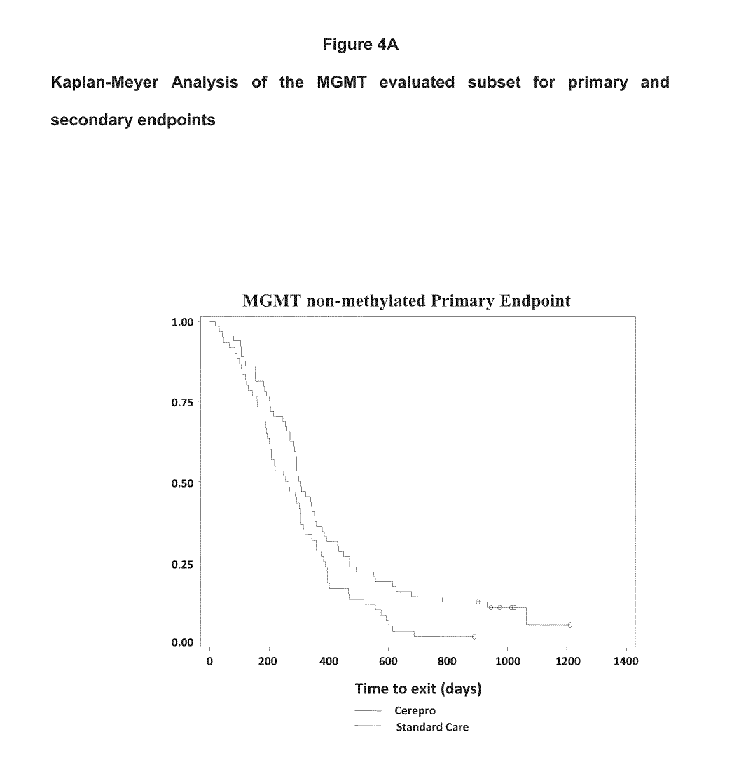

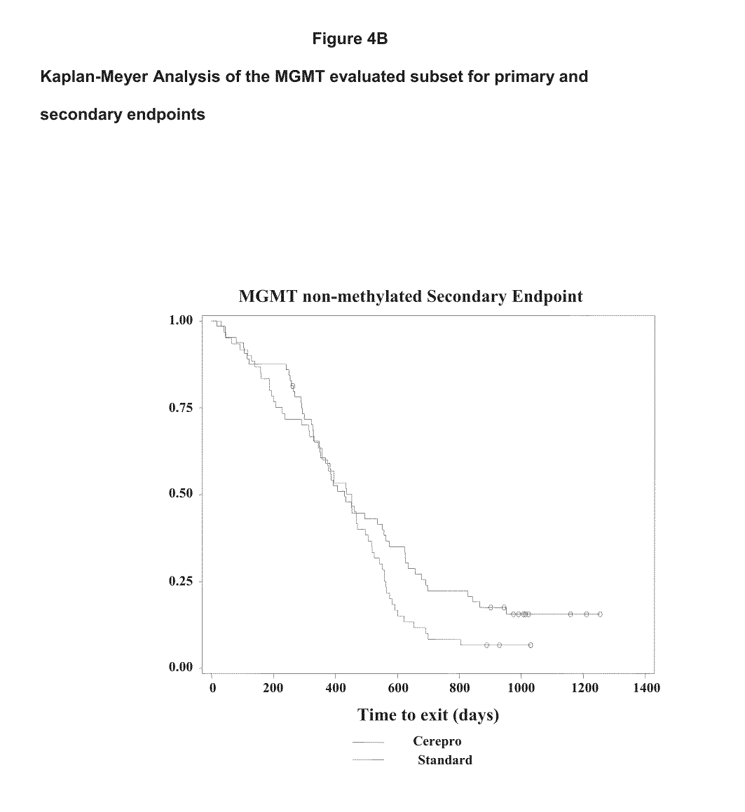

A subgroup analysis looked at the effect of SIT in patients in relation to MGMT promoter methylation status. SIT had a greater impact on the primary endpoint in this subgroup (HR=1·72, 95% Cl 1·15-2·56, p=0·008), (FIG. 4A) and the effect on time to all-cause mortality also increased, though still without statistical significance (HR=1·40, 95% Cl 0·92-2·12, p=0·112) (FIG. 4B.)

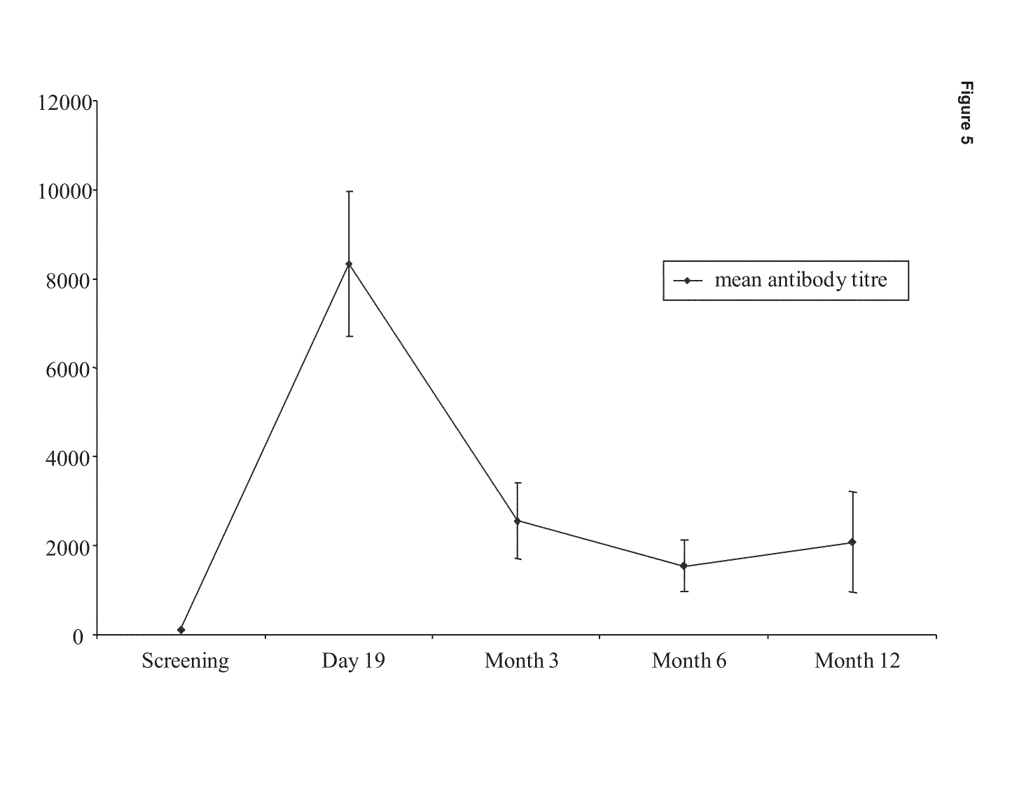

As treatment efficacy can potentially be influenced by neutralising antibodies these were screened for. Prior to treatment all patients had their antibody status assessed but a total of 46 patients (39%) in the Cerepro group and 37 patients (30%) in the standard care group had quantifiable antiadenoviral antibodies, presumably following environmental exposure. An additional 8 patients (7%) in the Cerepro group and 10 patients (8%) in the standard care group had detectable but nonquantifiable levels. By Day 19 there was a notable increase in the number of patients with detectable anti-adenoviral antibody levels following Cerepro treatment (84 patients [75%] with quantifiable and 14 patients [13%] with non-quantifiable levels) and in mean antibody titre (from 162-3706). Both the proportion of patients with anti-adenoviral antibodies and the mean antibody titre gradually declined over the subsequent 12 months (FIG. 5). In comparison the mean antibody titre remained relatively constant in patients undergoing Standard Care. In an analysis of treatment effect by baseline antibody status it was noticed that the effect of SIT appeared to be greater in patients with a higher titre of neutralising antibodies (Table 2)

TABLE 2
Baseline Antibody Titre and Efficacy of Sitimagene Ceradenovec

| | Primary Endpoint | | All Cause Mortality | |
|---|---|---|---|---|
| | Hazard | p-value vs | Hazard | p-value vs |
| Antibody | Ratio | Control | Ratio | Control |
| titre (n) | (95% CI) | Group | (95% CI) | Group |
| 0 (131) | 1.29 | .221 | 1.07 | .778 |
| | (.86, 1.93) | | (.68, 1.66) | |
| >0 (98) | 1.55 | .063 | 1.76 | .025 |
| | (.98, 2.45) | | (1.07, 2.87) | |
| >100 (47) | 2.17 | .047 | 1.89 | .116 |
| | (1.01, 4.64) | | (.85, 4.16) | |

It should be noted that these analyses were performed on the data set from March 2009, whereas other efficacy data reported herein are from the subsequent October 2009 data set.

(Patent application filed: Farries T and Eckland D. Patent “Cancer Therapy” published 2011 with international publication number WO 2011/036487 A1).

### Safety

In this open study, similar numbers of AEs were reported in the two groups, (96% SIT, 97% Standard Care). More patients in the SIT group reported a severe AE (65% versus 52%), and an AE considered related to study intervention (71% versus 41%). In all, 60% of SIT group patients versus 43% Standard Care group patients reported at least one SAE. Adverse events were assessed in time intervals after surgery; days 0-5, 5-19 (during ganciclovir) and after day 19. The events of concern occurred before day 19, the picture beyond this point being the events of other medication (temozolomide, radiotherapy) and of recurrent disease. Other than pyrexia (seen in 19% of SIT patients and 8% of Standard Care in days 0-5) the events were in the central nervous system. Cerebral ooedema was seen more commonly in SIT (6 vs 4 patients and 2 vs 0 patients, on days 0-5 and 5-19 respectively). Cerebral haematoma or haemorrhage was no more common in the SIT than Standard Care group. Hemiparesis was more common with SIT early after operation (15 vs 9 patients during days 0-5) but in the SIT group nine of these were worsening pre-operative findings; and one was noted to be caused by the surgery. Seven of the 15 noted their symptoms improved after the event. Seizures were notably increased in prevalence, but occurring only during days 5-19 (13 patients on SIT, 2 on Standard Care). Ganciclovir is reported to cause seizures.

Hyponotraemia was reported more frequently (9 vs 0 patients) on days 5-19—it should be noted, however, that most Standard Care patients will have left hospital for this time and would not have been subject to blood tests.

Few patients had measurable circulating vector DNA post operatively (Table 3), and the amount of vector DNA circulating declined rapidly with time.

TABLE 3
Biodistribution of Sitimagene Ceradenovec

| | | Day | Day | Day | Day | Month | Month |
|---|---|---|---|---|---|---|---|
| | Baseline | 1 | 2 | 5 | 19 | 3 | 6 |
| Quantifiable | 0 | 8 | 3 | 1 | 1 | 0 | 0 |
| Value n | 0 | 47 | 45 | 24 | 15 | 3 | 0 |
| Detected but | | | | | | | |
| below the limit of | | | | | | | |
| quantification n | | | | | | | |
| N | 80 | 112 | 110 | 108 | 99 | 84 | 67 |

No relationship between circulating vector DNA and adverse events could be found.

### Discussion

A major problem in the treatment of glioblastoma multiforme is that the tumour is protected from most systemic therapies^{23,24}. Thus our approach offers therapeutic possibilities that, using gene therapy, treat the tumour locally and also have the advantage of no systemic exposure of the gene and vector. Adenovirus is clinically safe and can be manufactured on large scale.

Furthermore, the AdvHSV-TK gene therapy has another safety feature: cell killing effect is only induced after the intravenous administration of a non-toxic prodrug, GCV, which is then converted by the adenoviral delivered HSV-TK enzyme into an active cytotoxic nucleoside analog, locally active. Promising clinical phase II trial results with this combination¹⁵ justified a phase III trial of AdvHSV-TK/GCV gene therapy for GBM.

This study was undertaken on a large cohort of patients, using AdvHSV-TK/GCV gene therapy to attempt to improve the outcome of patients with resectable, newly diagnosed glioblastoma. A positive effect on the primary composite endpoint was demonstrated. However, no statistically significant effect was shown for overall survival. The study is encouraging and the magnitude of the treatment effect seems valuable in relation to progress being made in the treatment of GBM. Importantly, the overall outcome including interpretation of the results raises some issues for the conduct of trials in this area.

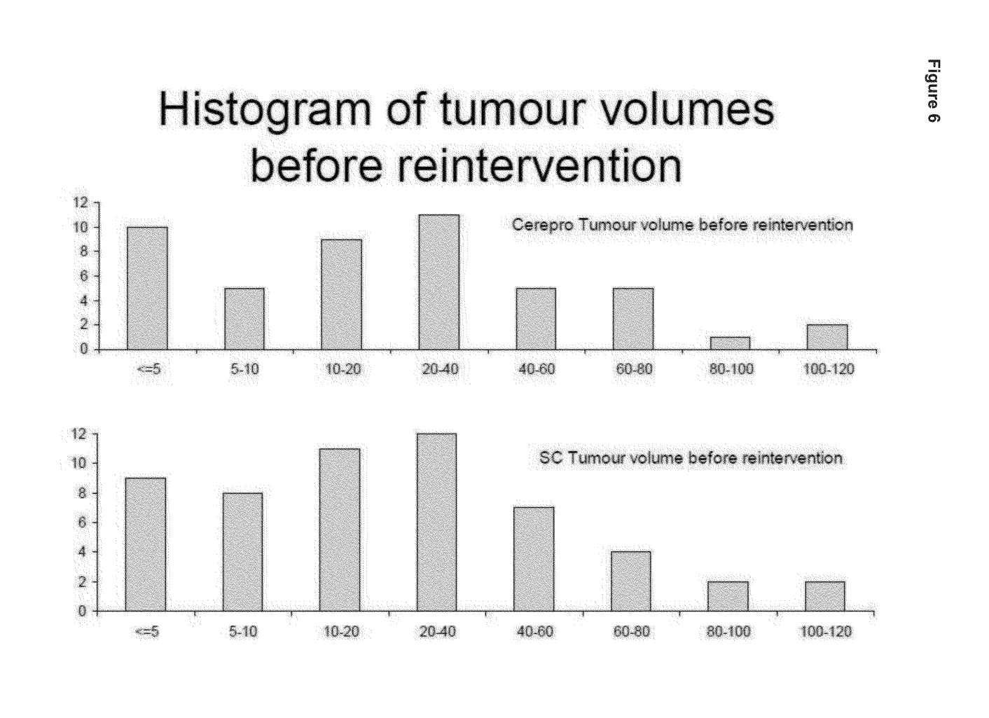

The nature of the treatment predicated the need for an open study. Blinding the treatment could have been achieved only by using multiple “neutral” intracerebral injections with saline which themselves would carry a risk for the control group patient. Alternatively, to inject a control adenoviral gene construct would also carry risk. Furthermore control patients would have to have received intravenous gangcyclovir to achieve comparability. Thus, in any open study with an endpoint as chosen here,—time to death or re-intervention—steps must be taken to exclude bias. Extensive analysis was performed of imaging and clinical characteristics at the time point when progression was determined and re-intervention indicated. A Re-Intervention Committee (RIC) blinded to treatment monitored the trial to assess whether a bias could be detected when re-intervention was called for. The RIC found that there was no bias when looking at the whole patient cohort. To obtain evidence that there was no bias in the assessment of the primary endpoint, the tumour sizes for both groups at the time of re-intervention were compared. As the sizes for both groups are nearly identical, it can be excluded that patients in the treatment arm were allowed to go longer until progression was determined or conversely that patients in the standard care arm were identified to have progressed earlier with smaller tumours, which is in accordance with the (blinded) RIC findings (FIG. 6).

The emergence of temozolamide a then new, effective effective treatment strategy relevant to both treatment arms while the study was progressing posed another challenge. The confounding pattern of temozolamide use rendered the initial statistical analysis strategy inappropriate.

Therefore, legitimately a post hoc analysis was undertaken, with the consequent risks of loss of control of alpha. Whilst the extent of this loss of control cannot be calculated, the analysis used the same population, the same endpoint and in principle, the same type of analysis. We therefore believe that the magnitude and significance of the treatment effect is a fair reflection of the treatment benefit, given the nature of the analyses undertaken, and the degree of statistical significance seen.

A Cox model with use of covariates in the analysis, namely age, baseline KPS, extent of resection, MGMT status and the use of temozolomide is justified, as each of these is known to have an important impact on the outcome^{2,4,25,26}, The imbalanced use of temozolamide in the control arm where more patients received it than in the gene therapy arm might be a random event or the result of bias in an open study design and this has to be acknowledged. In any case, unless an unlikely additive effect of temozolamide or a hypothetical positive interaction with gene therapy would be postulated without any supportive evidence, the imbalance of an active agent towards a control arm would rather have a negative statistical effect on the investigational agent.

This study was undertaken in an almost exclusively Caucasian population, across a number of European countries, and Israel. There seems to be no evidence that the disease of GBM is different in other ethnic groups, and these results should apply to all ethnic groups. Whilst the technology involved in the manufacture of gene therapy is complex, its administration in this setting is simple, and could be done at any neurosurgical centre. Also, AdHSV-TK/GCV treatment is given only once at the time of surgery and is therefore not very demanding for the patients. In future, repeated application might be possible to increase the treatment effect.

In this context the effect of SIT in patients whose tumours had non-methylated MGMT promoter is of interest. As it was impossible to be aware of the MGMT status of the tumours at the time of treatment, this could not have influenced the therapeutic decision. Further, the effect of temozolomide in the MGMT non-methylated patients is rather small²¹. Interestingly, the effect of SIT in this subgroup on the primary endpoint was highly significant although significance for overall survival was not seen. It should be noted, however, that statistical power within such subgroups inevitably lacks power.

The current report illustrates, that the selection of appropriate endpoints for clinical trials in GBM testing any intervention is problematic. As recognised by the RANO (response assessment in neuro-oncology) initiative¹ the frequently used progression free survival at six months (PFS6) has the potential to be confounded by “pseudoprogression”, especially in the setting of local therapies where new criteria to define progression are emerging²⁷Overall survival is certainly a strong endpoint but for newly diagnosed tumours it might be unsuitable to capture meaningful treatment effects for the first stage of the disease because of uncontrolled therapy at recurrence²⁸.

The apparent increased efficacy with increased baseline titre of neutralising antibodies is perhaps surprising. It might be expected that neutralising antibodies would reduce the extent of transduction, and hence the treatment effect, but this does not seem to be the case in this setting, likely as, adenoviruses bind quickly to their cellular receptors and the presence of neutralising antibodies may not necessarily significantly reduce the tranduction efficacy in the walls of the tumour cavity. It may also be that the presence of neutralising antibodies is a marker of a patient with a more robust immune system, and part of the bystander effect of SIT may be to initiate or enhance an immune response against the tumour. Such immunologically mediated bystander effects have been previously suggested^{,29-31} but much of what happens immunologically in situ in and around a tumour remains speculative given the many direct immunomodulary capacities of glioma cells^{9,32}.

The overall benefit seen, albeit from a post hoc analysis, is of the same order as seen with temozolomide, though this remains to be proven in a direct comparison. The safety profile of SIT is mainly concerned with hemiparesis (which was transient in many cases), hyponatraemia and seizures. Perhaps, surprisingly, for a locally injected therapy, there was no excess of cerebral haemorrhage or haematoma. In the context of the disease and prognosis, these effects are acceptable. Overall, the risk benefit ratio of this treatment appears positive.

Since this study was completed, no further phase III gene therapy trial has been published. The field of gene therapy for GBM, is nevertheless still evolving rapidly and becomes increasingly diversified as has recently been comprehensively summarized³³. There is a broad variety of viruses with different concepts like oncolysis, or pro-drug conversion/suicide gene and issues like replication competence or specific targeting that expand vector complexity. Many of these studies which are in early clinical trials are based on local treatment strategies like intratumoural or perilesional application. They will face the same issues of endpoint, imaging based assessments and biomarker associated subgroup efficacy as this study. All these issues, especially a statistically adapted analysis plan that accounts for prespecified or preconceived biomarker based subgroup analysis would ensure large enough sample sizes so that statistically meaningful results could be obtained.

In summary the ASPECT trial results reflect a significant advance from the earlier retrovirus based phase III study for newly diagnosed glioblastoma where absolutely not effect was seen⁷. With the significant efficacy result seen here for the primary endpoint and a signal for overall survival in the subgroup of MGMT methylated patients this completed phase III trial is encouraging step forward for the field of surgically applied, virally mediated local gene therapy.

### REFERENCES

1. Wen P Y, Kesari S. Malignant gliomas in adults. N Engl J Med 2008; 359(5):492-507.
2. Stummer W, Reulen H J, Meinel T, et al. Extent of resection and survival in glioblastoma multiforme: identification of and adjustment for bias. Neurosurgery 2008; 62(3):564-76; discussion 564-76.
3. Sanai N, Polley M Y, McDermott M W, Parsa A T, Berger M S. An extent of resection threshold for newly diagnosed glioblastomas. J Neurosurg 2011; 115(1):3-8.
4. Stupp R, Mason W P, van den Bent M J, et al. Radiotherapy plus concomitant and adjuvant temozolomide for glioblastoma. N Engl J Med 2005; 352(10):987-96.
5. Westphal M, Hilt D C, Bortey E, et al. A phase 3 trial of local chemotherapy with biodegradable carmustine (BCNU) wafers (Gliadel wafers) in patients with primary malignant glioma. Neuro Oncol 2003; 5(2):79-88.
6. Kunwar S, Chang S, Westphal M, et al. Phase III randomized trial of CED of IL13-PE38QQR vs Gliadel wafers for recurrent glioblastoma. Neuro Oncol 2010; 12(8):871-81.
7. Rainov N G. A phase III clinical evaluation of herpes simplex virus type 1 thymidine kinase and ganciclovir gene therapy as an adjuvant to surgical resection and radiation in adults with previously untreated glioblastoma multiforme. Hum Gene Ther 2000; 11(17):2389-401.
8. Moolten F L. Drug sensitivity (“suicide”) genes for selective cancer chemotherapy. Cancer Gene Ther 1994; 1(4):279-87.
9. Bi W L, Parysek L M, Warnick R, Stambrook P J. In vitro evidence that metabolic cooperation is responsible for the bystander effect observed with HSV tk retroviral gene therapy. Hum Gene Ther 1993; 4(6):725-31.
10. Asklund T, Appelskog I B, Ammerpohl O, et al. Gap junction-mediated bystander effect in primary cultures of human malignant gliomas with recombinant expression of the HSVtk gene. Exp Cell Res 2003; 284(2):185-95.
11. Soudais C, Laplace-Builhe C, Kissa K, Kremer E J. Preferential transduction of neurons by canine adenovirus vectors and their efficient retrograde transport in vivo. FASEB J 2001; 15(12):2283-5.
12. Thomas C E, Edwards P, Wickham T J, Castro M G, Lowenstein P R. Adenovirus binding to the coxsackievirus and adenovirus receptor or integrins is not required to elicit brain inflammation but is necessary to transduce specific neural cell types. J Virol 2002; 76(7):3452-60.
13. Sandmair A M, Loimas S, Puranen P, et al. Thymidine kinase gene therapy for human malignant glioma, using replication-deficient retroviruses or adenoviruses. Hum Gene Ther 2000; 11(16):2197-205.
14. Puumalainen A M, Vapalahti M, Agrawal R S, et al. Beta-galactosidase gene transfer to human malignant glioma in vivo using replication-deficient retroviruses and adenoviruses. Hum Gene Ther 1998; 9(12):1769-74.
15. Immonen A, Vapalahti M, Tyynela K, et al. AdvHSV-tk gene therapy with intravenous ganciclovir improves survival in human malignant glioma: a randomised, controlled study. Mol Ther 2004; 10(5):967-72.
16. Langford G, Dayan A, Yla-Herttuala S, Eckland D. A preclinical assessment of the safety and biodistribution of an adenoviral vector containing the herpes simplex virus thymidine kinase gene (Cerepro) after intracerebral administration. J Gene Med 2009; 11(6):468-76.
17. Vlassenbroeck I, Califice S, Diserens A C, et al. Validation of real-time methylation-specific PCR to determine O6-methylguanine-DNA methyltransferase gene promoter methylation in glioma. J Mol Diagn 2008; 10(4):332-7.
18. Brandsma D, Stalpers L, Taal W, Sminia P, van den Bent M J. Clinical features, mechanisms, and management of pseudoprogression in malignant gliomas. Lancet Oncol 2008; 9(5):453-61.
19. Lindholm L H, Ibsen H, Dahlof B, et al. Cardiovascular morbidity and mortality in patients with diabetes in the Losartan Intervention For Endpoint reduction in hypertension study (LIFE): a randomised trial against atenolol. Lancet 2002; 359(9311):1004-10.
20. Whitehead J. Monotherapy trials: sequential design. Epilepsy Res 2001; 45(1-3):81-7; discussion 89-91.
21. Hegi M E, Diserens A C, Gorlia T, et al. MGMT gene silencing and benefit from temozolomide in glioblastoma. N Engl J Med 2005; 352(10):997-1003.
22. Schmoor C, Schumacher M. Effects of covariate omission and categorization when analysing randomized trials with the Cox model. Stat Med 1997; 16(1-3):225-37.
23. Ram Z, Culver K W, Oshiro E M, et al. Therapy of malignant brain tumours by intratumoural implantation of retroviral vector-producing cells. Nat Med 1997; 3(12):1354-61.
24. Pulkkanen K J, Yla-Herttuala S. Gene therapy for malignant glioma: current clinical status. Mo Ther 2005; 12(4):585-98.
25. Gorlia T, van den Bent M J, Hegi M E, et al. Nomograms for predicting survival of patients with newly diagnosed glioblastoma: prognostic factor analysis of EORTC and NCIC trial 26981-22981/CE.3. Lancet Oncol 2008; 9(1):29-38.
26. Mirimanoff R O, Gorlia T, Mason W, et al. Radiotherapy and temozolomide for newly diagnosed glioblastoma: recursive partitioning analysis of the EORTC 26981/22981-NCIC CE3 phase III randomized trial. J Clin Oncol 2006; 24(16):2563-9.
27. Vogelbaum M A, Jost S, Aghi M K, et al. Application of novel response/progression measures for surgically delivered therapies for gliomas: Response Assessment in Neuro-Oncology (RANO) Working Group. Neurosurgery 2012; 70(1):234-43; discussion 243-4.
28. van den Bent M J, Vogelbaum M A, Wen P Y, Macdonald D R, Chang S M. End point assessment in gliomas: novel treatments limit usefulness of classical Macdonald's Criteria. J Clin Oncol 2009; 27(18):2905-8.
29. Floeth F W, Aulich A, Langen K J, Burger K J, Bock W J, Weber F. MR imaging and single-photon emission CT findings after gene therapy for human glioblastoma. AJNR Am J Neuroradiol 2001; 22(8):1517-27.
30. Floeth F W, Shand N, Bojar H, et al. Local inflammation and devascularization—in vivo mechanisms of the “bystander effect” in VPC-mediated HSV-Tk/GCV gene therapy for human malignant glioma. Cancer Gene Ther 2001; 8(11):843-51.
31. Niculescu-Duvaz I, Springer C J. Introduction to the background, principles, and state of the art in suicide gene therapy. Mol Biotechnol 2005; 30(1):71-88.
32. Albesiano E, Han J E, Lim M. Mechanisms of local immunoresistance in glioma. Neurosurg Clin N Am 2010; 21(1):17-29.
33. Murphy A M, Rabkin S D. Current status of gene therapy for brain tumours. Transl Res 2012.

We claim:

1. In a method of treating glioblastoma in a human by surgical resection and temozolomide, an improvement comprising:
b. administering locally about the surgical resection site after surgical resection, about 1×10¹² particles of replication-deficient adenovirus serotype 5 with E1 and partial E3 deletions, and containing the cDNA for HSV-tk which is able to phosphorylate ganciclovir, then
c. administering gancyclovir 5 mg/kg i.v., twice daily.

2. The method of claim 1, where said glioblastoma is amenable to complete resection.

3. The method of claim 1, where said glioblastoma is neither bi-hemispheric nor multifocal.

4. The method of claim 2, where said glioblastoma is neither bi-hemispheric nor multifocal.

5. The method of claim 1, wherein said replication-deficient adenovirus is administered as a series of injections using a blunt needle advanced up to about 2 cm (tissue depth permitting).

6. The method of claim 5, entailing from about 30 to about 70 injections.

7. The method of claim 5, wherein said series of injections are into the wall of the resection cavity at the end of the complete resection.

8. The method of claim 5, wherein a plurality of said injections administer about 100 μl per injection site.

9. The method of claim 5, wherein a plurality of said injections administer sufficient adenovirus suspension to produce cavitations visible on MRI.

10. The method of claim 1, wherein said administering gancyclovir begins at least about five days after administering said adenovirus.

11. The method of claim 1, further comprising:
a. before administering said adenovirus, assaying said human to determine said human's MGMT promoter methylation status.

12. The method of claim 11, wherein said human's MGMT promoter methylation status is non-methylated.

13. The method of claim 1, further comprising:
a. before administering said adenovirus, assaying said human to determine said human's anti-adenovirus antibody titer.

14. The method of claim 13, wherein said anti-adenovirus antibody titer is high enough to be quantifiable.

15. The method of claim 13, said adenovirus administered in an amount sufficient to increase said human's antibody titer.

16. (canceled)

17. The method of claim 1, wherein said administration extends the time to death or re-invention for said human.

18. The method of claim 1, said adenovirus formulated in 5 mM Hepes buffer pH 7.8 containing about 20% (v/v) glycerol.

19. The method of claim 1, said about 1×10¹² particles of replication-deficient adenovirus administered in a volume of about 10 ml of vehicle.

20. The method of claim 11, wherein said vehicle comprises physiological saline.

21. A method comprising:
a. treating glioblastoma in a human by surgical resection, and then
c. administering locally about the surgical resection site after surgical resection, about 1×10¹² particles of replication-deficient adenovirus serotype 5 with E1 and partial E3 deletions, and containing the cDNA for HSV-tk which is able to phosphorylate ganciclovir, and then
d. administering to said human gancyclovir 5 mg/kg i.v., twice daily, and
e. administering to said human temozolomide.

22-40. (canceled)


SEQUENCE LISTING (3 sequences)

SEQ ID NO: 1 - DNA, 24 bp, Adenovirus type 37
ggctaactagagaacccactgctt

SEQ ID NO: 2 - DNA, 21 bp, Adenovirus type 37
aagccatacgcgcttctacaa

SEQ ID NO: 3 - DNA, 22 bp, Adenovirus type 37
tggcgtgaaactcccgcacctc